(19) United States
(12) Patent Application Publication
(10) Pub. No.: US 2023/0061352 A1
(43) Pub. Date: Mar. 2, 2023

(54) DRUG DELIVERY DEVICE

(71) Applicants: Anders William James Sideris, Bondi (AU); Kyle Andrew Thomson, Zetland (AU); Thomas Ernest Havas, Bondi Junction (AU)

(72) Inventors: Anders William James Sideris, Bondi (AU); Kyle Andrew Thomson, Zetland (AU); Thomas Ernest Havas, Bondi Junction (AU)

(21) Appl. No.: 17/796,348

(22) PCT Filed: Jan. 29, 2021
(86) PCT No.: PCT/AU2021/050059

§ 371 Date: Jul. 29, 2022

(30) Foreign Application Priority Data
Jan. 31, 2020 (AU) 2020900258

(51) Int. Cl. A61M 31/00; A61K 9/00; A61K 31/722; A61K 31/765

(52) U.S. Cl. CPC A61M 31/002; A61K 9/006; A61K 31/722; A61K 31/765

(57) ABSTRACT

A layered drug delivery device which includes a polymeric tissue interface layer and a polymeric backing layer. The polymeric tissue interface layer includes at least one ther-apeutic agent.

### FIELD OF THE INVENTION

The present invention relates to drug delivery devices and, in particular, to layered drug delivery devices that can be located against body tissue for sustained and controlled delivery of one or more therapeutic agents to, for example, aid wound healing and/or provide pain relief.

### BACKGROUND OF THE INVENTION

Any reference herein to known prior art does not, unless the contrary indication appears, constitute an admission that such prior art is commonly known by those skilled in the art to which the invention relates, at the priority date of this application.

Recovery from surgery is often painful and wounds can take a long time to heal. Pain and healing time can be exacerbated by postoperative bleeding. In otolaryngology for example, the most commonly performed operation is the tonsillectomy. Oropharyngeal pain remains the primary cause of morbidity in the post-tonsillectomy patient and has been linked to decreased oral intake, dysphagia, dehydration and weight loss. Furthermore, postoperative bleeding as a result of these surgeries has been linked to poor wound healing and low-grade infection. In extreme cases, these postoperative bleeds can be severe and lead to hypovolaemic shock and death.

Typical methods of pain management include intraoperative use of local anaesthetic agents, paracetamol, opiate medications, and non-steroidal anti-inflammatory (NSAIDS). However, each of these have their limitations. Intraoperative use of local anaesthetic agents are effective postoperatively but only last short periods of time (up to 8 hours), paracetamol alone is not effective, systemic opiate medications cause sedation and respiratory depression, particularly in paediatric patients, and the use of NSAIDs has been linked to more severe bleeding when it occurs.

In addition, poor medication compliance compounds the difficulties with pain management and wound healing. Poor compliance is linked to inadequate pain control, which causes significant morbidity for the patient and unnecessary additional cost to the healthcare system. In particular, representation to local doctors (General Practitioners) and emergency departments for poorly controlled pain or oral intake continue to occur regardless of analgesia regimen.

The present invention seeks, according to one aspect, to address the difficulties associated with the post-operative pain management, wound healing and medication compliance.

### SUMMARY OF THE INVENTION

In one broad form, the present invention provides a layered drug delivery device including: a polymeric tissue interface layer including at least one therapeutic agent; and a polymeric backing layer.

In some forms, the tissue interface layer comprises a biopolymer and a second polymer. In some forms, the biopolymer is mucoadhesive. In some forms, the biopolymer is chitosan. In some forms, the second polymer is polycaprolactone.

In some forms, wherein the backing layer, or a sublayer thereof, is configured to prohibit diffusion of therapeutic agent therethrough. In some forms, the backing layer includes any one or a combination of polycaprolactone, polysiloxane, PLLA, PLGA, and/or a copolymer of PLLA and PLGA.

In some forms, the layered drug delivery device further includes one or more additional release layers sandwiched between the tissue interface layer and the backing layer, each additional release layer including: a polymeric spacing sublayer; and a polymeric dosage sublayer including at least one therapeutic agent, wherein the sublayers of each additional release layer are ordered such that each spacing sublayer is closer to the tissue interface layer than its respective dosage sublayer.

In some forms, the spacing sublayer of each additional release layer comprises any one or a combination of PLLA, PLGA and/or a copolymer PLLA and PLGA. In some forms, the dosage sublayer of each additional release layer comprises a biopolymer and a second polymer. In some forms, in the dosage sublayer, the biopolymer is chitosan. In some forms, in the dosage sublayer, the second polymer is polycaprolactone.

In some forms, one or more of the additional release layers are perforated. In some forms, the tissue interface layer is perforated.

In some forms, the layered drug delivery device is convex at the tissue interface layer side and concave at the backing layer side. In some forms, the device is shaped to be located against a wall of the tonsillar fossa.

In some forms, the layered drug delivery device is patch or the like to be located against tissue at a treatment area.

In some forms, the layered drug delivery device is biodegradable. In some forms, the backing layer is configured to degrade more slowly than any other layer in the device.

In some forms, the layered drug delivery device is substantially porous. In some examples, pore sizes for the device are in the range of 200 nm to 600 nm. In some examples, pore sizes for the device are in the range of 6 μm to 60 μm. In some examples pore sizes for the device are in the range of 60 μm to 120 μm. Typically, pore sizes are configured dependent on respective layer thickness (i.e. small enough so as not to fully penetrate the respective layer in which they are present). In some forms, the backing layer or a sublayer thereof is not substantially porous.

In some forms, the at least one therapeutic agent includes an anesthetic agent. In some forms, the at least one therapeutic agent includes a biomolecule. In some forms, the at least one therapeutic agent includes an antimicrobial agent. In some forms, the at least one therapeutic agent includes an antifungal agent. In some forms, the at least one therapeutic agent includes an anti-viral agent. In some forms, the at least one therapeutic agent includes a chemotherapeutic agent. In some forms, the at least one therapeutic agent includes an immune modulation agent. In some forms, the at least one therapeutic agent includes a cell growth or differentiation promoting agent. In some forms, the at least one therapeutic agent includes a steroidal or non-steroidal anti-inflammatory.

In some forms, the tissue interface layer is substantially hydrophilic. In some forms, the backing layer is substantially hydrophobic. In some forms, the layers thereof are continuous. In some forms, the layers thereof are substantially planar.

In a further broad form, the present invention provides, a layered drug delivery device including: a polymeric tissue interface layer including at least one therapeutic agent; a polymeric backing layer; and one or more additional release layers sandwiched between the tissue interface layer and the backing layer, each additional release layer including: a polymeric spacing sublayer; and a polymeric dosage sublayer including at least one therapeutic agent, wherein the sublayers of each additional release layer are ordered such that each spacing sublayer is closer to the tissue interface layer than its respective dosage sublayer.

In some forms, the device includes at least two additional release layers.

In some forms, the tissue interface layer is formed of a polymer matrix with ther-apeutic agent incorporated therein. In some forms, the dosage sublayer(s) is/are formed of a polymer matrix with therapeutic agent incorporated therein. In some forms, the tissue interface layer comprises a polymer matrix formed of a blend of two or more polymers. In some forms, the tissue interface layer is formed of a blend of chitosan and PCL. In some forms, each dosage sublayer comprises a polymer matrix formed of a blend of two or more polymers. In some forms, the dosage sub-layer(s) is/are formed of a blend of chitosan and PCL.

In some forms, the spacing sublayer(s) is/are configured to slow or delay release of therapeutic agent from the dosage sublayers. In some forms, the spacing sublayer(s) is/are formed of a copolymer of PLLA and PLGA.

In some forms, the backing layer is configured to substantially prohibit diffusion or permeation of therapeutic agent therethrough. In some forms, the backing layer includes a layer of PCL. In some forms, the backing layer includes a sublayer formed of copolymer of PLLA and PLGA, and a sublayer formed PCL, the PCL sublayer being the outermost layer, furthest from the tissue interface layer.

In some forms, the device is a patch configured for securement in the oropharynx. In some forms, the device includes mounting portions to facilitate securement to a treatment site.

In some forms, the tissue interface layer comprises two or more sequentially cast polymeric sublayers that interpenetrate one another. In some forms, the sequentially cast sublayers of the tissue interface layer comprise chitosan. In some forms, the neighboring sublayers interpenetrate one another by about 25-35%, as proportionate to their width.

In some forms, one or more intermediate layers are sandwiched between the tissue interface layer and the backing layer. In some forms, each intermediate layer comprises a polymer blend of two or polymers. In some forms, one or more of the intermediate layers include at least one therapeutic agent.

In a further broad form, the present invention provides a method of treating an oropharyngeal wound, the method including the steps of: securing a device provided in any of the forms described herein against the wound. In a further broad from, the present invention provide a method of treating a tonsillectomy wound, the method including the steps of: securing a device as provided in any of the forms described herein against the wound.

In a further broad form, the present invention relates to use of a device as provided in any one of the above forms, in the treatment of an oropharyngeal wound or a tonsillectomy wound.

In a further broad form, the present invention provides a tissue interface for a drug delivery device, the tissue interface comprising two or more sequentially cast polymeric layers that interpenetrate one another. In some forms, the polymeric layers are chitosan layers.

### BRIEF DESCRIPTION OF THE DRAWINGS

Embodiments of the present invention will be described in further detail with reference to the drawings from which further features, embodiments and advantages may be taken, and in which:

FIG. 1 is a side perspective view of a layered drug delivery device according to one example of the invention;

FIG. 2 is portion A from FIG. 1 enlarged, showing the layered structure of the device;

FIG. 3 is portion B from FIG. 2 enlarged, showing therapeutic agent loading in some of the layers;

FIG. 4 is a diagram of the oral cavity, showing typical placement of the device of FIG. 1 in the tonsillar fossa;

FIG. 5 is a schematic diagram illustrating suture positioning for securement of the device according to one example;

FIG. 6 is a schematic diagram illustrating mounting portions or ‘islands’ in the device according to one example;

FIG. 7 is a chromatogram illustrating detection of bupivacaine mass across a range of masses and a highly specific assay confirming bupivacaine mass with no other signal detected indicating a complete, intact bupivacaine molecule;

FIG. 8 is a chromatogram illustrating detection of lignocaine mass across a range of masses and a highly specific assay confirming bupivacaine mass with no other signal detected indicating a complete, intact lignocaine molecule;

FIG. 9 is a line graph representing the bupivacaine levels detected by LCMS method in porcine tonsillar tissue taken at necropsy at 0, 48, 120, 240 and 336 hrs;

FIG. 10 is a line graph representing the lignocaine levels detected by LCMS method in porcine tonsillar tissue taken at necropsy at 0, 48, 120, and 240 hrs;

FIG. 11 is a line graph showing the cumulative percentage release of bupivacaine and lignocaine detected by the described LCMS method in porcine tonsillar tissue taken at necropsy at 0, 48, 120, 240 and 336 hrs;

FIG. 12 is a line graph representing the bupivacaine levels detected by the described LCMS method in porcine lymph tissue taken from the anterior jugular chain at necropsy at 0, 48, 120, 240 and 336 hrs, the bupivacaine levels expressed in nanograms per milligram of lymph tissue;

FIG. 13 is a line graph representing the lignocaine levels detected by the described LCMS method in porcine lymph tissue taken from the anterior jugular chain at necropsy at 0, 48, 120, 240 and 336 hrs, the lignocaine levels expressed in nanograms per milligram of lymph tissue;

FIG. 14 is a line graph representing the lignocaine levels detected by the described LCMS method in porcine serum taken from the internal jugular vein at 0, 1, 2, 4, 24, 48, 72, 120, the lignocaine levels expressed in nanograms per millilitre of serum;

FIG. 15 is a line graph representing the bupivacaine levels detected by the described LCMS method in porcine serum taken from the internal jugular vein at 0, 1, 2, 4, 24, 48, 72, 120, the bupivacaine levels expressed in nanograms per millilitre of serum;

FIG. 16 is a scanning electron microscopy image of the device in one example, at ×100 magnification;

FIG. 17 is a scanning electron microscopy image of the device according to one example, at ×250 magnification;

FIG. 18 demonstrates an intraoral view of the device described according to one example, placed in a porcine tonsillectomy wound at time of placement;

FIG. 19 demonstrates an intraoral view of the device described according to one example, sutured to the porcine tonsillectomy wound 5 days from implantation;

FIGS. 20 to 27 show histology of tissues harvested from porcine tonsillectomy samples at necropsy at time 48, 120, 240 and 336 hours, demonstrating the tissue responses at the device-tissue interface;

FIGS. 28 to 30 respectively show chitosan films under light microscopy prior to immer-sion in salivary enzyme at 1.25×, 2×, and 4× magnification;

FIGS. 31 to 33 respectively show chitosan films under light microscopy 48 hours from immersion in salivary enzyme at 1.25×, 2×, 4× magnification, illustrating degradation of the polymer;

FIG. 34 shows a chitosan layer constructed in one solvent cast and a chitosan layer constructed in two solvent casts, the latter showing an interpenetrating or inter-melding phase (30) at the overlap region;

FIG. 35 illustrates a schematic of an extended oropharyngeal resection of a tonsillar cancer; and

FIG. 36 illustrates a schematic of an oropharyngeal resection defect to which the shape and contour of the device may be customised.

### DETAILED DESCRIPTION OF PREFERRED EMBODIMENTS

Embodiments of the invention provide a layered drug delivery device to provide controlled and sustained delivery of therapeutic agents to surgical and other treatment sites. The device may have a range of applications, including, but not limited to, applications in pain management, wound healing, and/or the treatment of tumours, in-flammation, and infection. Embodiments of the device have particular application for tonsillectomy patients, providing a means to deliver local anesthetic agents post-tonsillectomy, promote post-tonsillectomy wound healing and/or prevent post-tonsillectomy infection and/or bleeding.

Embodiments of the device include a polymeric tissue interface layer that includes at least one therapeutic agent, and a backing (or support) layer. The backing (or support) layer is also typically polymeric. The tissue interface layer is that which, in use, is located against tissue to be treated. The tissue interface layer may comprise one or more polymers and one or more sublayers. In some examples, the tissue interface layer comprises two or more polymers. In one example, the tissue interface layer comprises a biopolymer and a second polymer. For example, the tissue interface layer may comprise a polymer matrix formed of a mixture/blend of a biopolymer and second polymer, with a therapeutic agent embedded/incorporated therein.

To facilitate adhesion of the device to mucosal tissue/sites, the tissue interface layer may be mucoadhesive. Typically, a biopolymer of the tissue interface layer is mucoadhesive and may be, for example chitosan. One example of tissue interface layer, is formed of a blend of chitosan and polycaprolactone (PCL).

In another example, the tissue interface layer may be formed of multiple sequentially cast sublayers of a polymer. For example, in one form, the tissue interface layer comprises sequentially cast sublayers of chitosan. In one form, the sublayers of chitosan overlap/inter-meld/inter-penetrate one another. In one example they interpenetrate by about 25-35% (as a proportion of their width). An example method for providing inter-melding/overlapping of layers is described by EXAMPLE 3. In one example, the tissue interface layer comprises 4 sequentially cast layers of chitosan that interpenetrate one another (i.e. with 3 intermelded/overlapping phases).

For delivery of therapeutic agent from the tissue interface layer to the treatment site, therapeutic agent typically diffuses to the treatment area and/or is released as the polymer matrix of the tissue interface layer degrades. The backing layer, or a sublayer thereof, is generally configured to substantially prohibit diffusion or permeation of therapeutic agent therethrough, so as to substantially prevent therapeutic agent escaping/progressing to tissue/areas other than the treatment site. The backing layer is typically formed of polymer(s) that degrade more slowly than those of the tissue interface layer (to avoid loss of therapeutic agent away from the treatment site). In some examples, the backing layer may include any one or a combination of PCL, polysiloxane, Poly-l-lactide acid (PLLA), poly-l-glycolic acid (PLGA), and/or a copolymer of PLLA and PLGA. The backing layer may be produced, in some examples, in accordance with the methods as described in EXAMPLE 1 or 5.

Generally, the layered drug delivery device further includes one or more additional release layers sandwiched between the tissue interface layer and the backing layer. Each additional release layer includes a polymeric spacing sublayer and a polymeric dosage sublayer that includes at least one therapeutic agent. The sublayers of each additional release layer are ordered such that each spacing sublayer is closer to the tissue interface layer than its respective dosage sublayer. Each spacing sublayer acts to slow or delay release of therapeutic agent from its respective dosage sublayer. For example, in use, once therapeutic agent is released from the tissue interface layer the spacing sublayer provides a temporary barrier that slows or delays release from the adjacent dosage sublayer. To progress to the treatment area, therapeutic agent from the dosage sublayer typically either diffuses over time across the spacing sublayer and/or is released once the spacing layer sufficiently degrades.

In some examples, the spacing sublayer of each additional release layer comprises any one or a combination of PLLA, PLGA and/or a copolymer PLLA and PLGA. The dosage sublayer of each additional release layer may be similar to the tissue interface layer, and may comprise two or more polymers, like for example, a biopolymer and a second polymer. In one example, in the dosage sublayer(s), the biopolymer may by chitosan and the second polymer may be PCL. In other examples, the dosage sublayer(s) may only comprise a single polymer, and may be formed, for example, principally of chitosan. In some examples, the spacing and dosage sublayer(s) are each substantially planar continuous polymer matrices. In some examples, the additional release layers an/dor their sublayers may be fabricated in accordance with the methods as described in EXAMPLE 1 or 4.

It will be appreciated that the drug delivery profile and/or degradation profile of the device can be modified/configured by the selection of the polymers that form each layer/sublayer. Thus, the device release kinetics can be pre-engineered/configured for a specific indication/application. For example, it will be appreciated that different polymer combinations/compositions will have different properties e.g. rates of layer degradation, drug release profiles, diffusion characteristics. The device may thus be configured for application in different environments within the body. For example, the polymer/layer composition may be configured for degradation within the environment of oropharynx, e.g. by salivary enzymes, at the pH of the oropharynx (pH 4.0-6.0) or Oral cavity (pH 6.0-8.0), etc.

It will be appreciated that the polymeric layers/sublayers of the devices as described herein each may be formed of one or more polymers, copolymers and/or polymer composite materials. It will also be appreciated that alayer or sublayer may produce by sequentially cast layers.

In addition to those already mentioned, suitable polymers that may imple-mented in the device include, but are not limited to, marine collagen, alginate, xanthan gum, cellulose, polydioxanone, polylactinone, polylactin, poloxamer, polyrthoesters, polyanhydride, poly(ethylene-co-vinyl acetate), poly(methyl methacrylate), poly(vinyl alco-hol), poly(N-vinyl pyrrolidone), poly(acrylic acid), poly(2hydroxy ethyl methacrylate), polyacrylamide, poly(methacrylic glycol), poly(ethelene glycol).

It will also be appreciated that other parameters may be adjusted in seeking to modify/configure the drug delivery profile including, but not limited to, the thickness of each layer/sublayer, the porosity of the layers/sublayers, the degree of overlap/interpenetration/inter-melding between layers and/or their sublayers, layer/sublayer biodegradability, and/or the number of additional release layers that are sandwiched between the tissue interface layer and the backing layer.

In respect to the degree of overlap/interpenetration/inter-melding between layers and/or their sublayers this can provides advantages in that:
There are no layer-layer delamination failures;
There is a smoother drug release profile i.e. less like repeated dosing in conventional administration schedules of a therapeutic, and more like a constant administration of select amounts. This may be desirable in some circumstances and can lead to a shorter treatment time. A smoother release profile limits short term (potentially toxic) bursts of therapeutic agent experienced by the tissue and cells (fibroblasts/epithelial/etc) at the device-tissue interface. This can help to achieve rapid and quality wound repair tissue; and/or
Improved degradation profiles. Similarly, a more consistent degradation profile provides more predictable outcomes for the devices, and less failures during its administration.

A device having initial layer structures on the tissue interface side that have differing boundary phases (i.e. intermelded phases), instead of strict layer-to-layer interfaces, assists proliferative cells during wound healing. Cells such as fibroblasts can more readily penetrate the devices structure. These cells migrate faster to fill the traditional wound void more rapidly and require fewer numbers to line the wound cavity. In doing so less tissue is created in the healing process of healing that subsequently requires further remodelling afterwards, such as those associated with the wound plug etc.

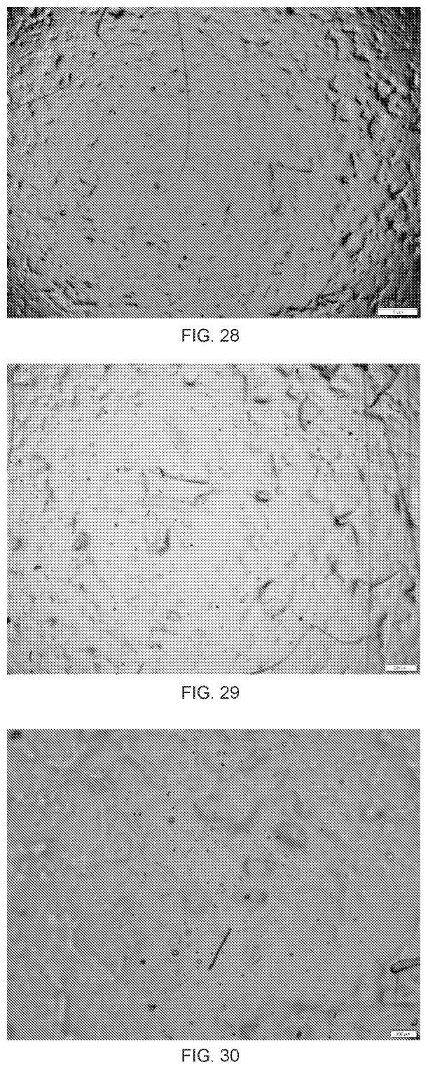

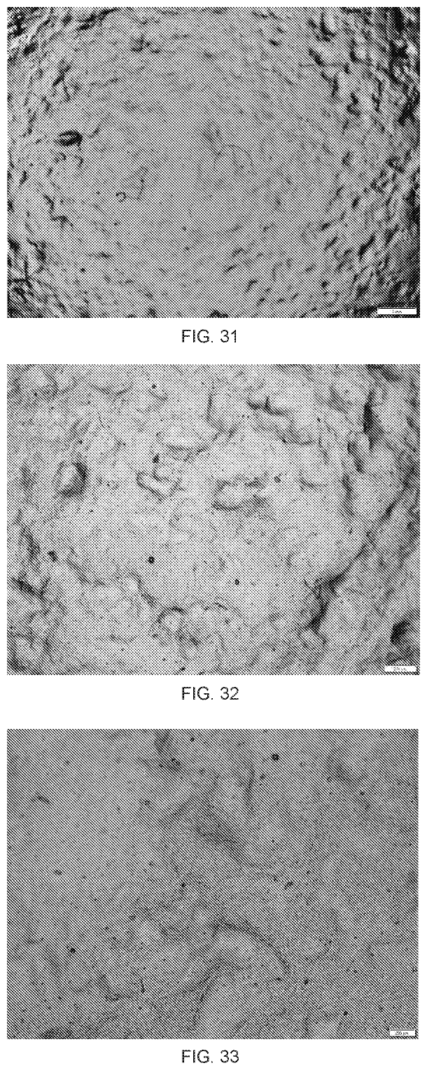

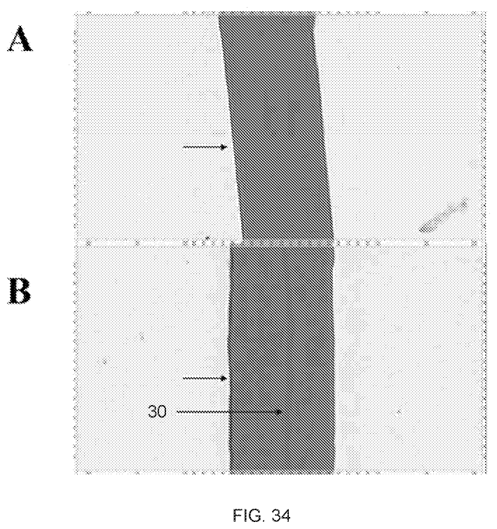

Typically, during casting, solvent characteristics can be configured to allow layers (e.g. of chitosan) to penetrate pre-existing layers during fabrication creating an over-lapping phase rather than a hard layer-layer interface. The depth of over-lap or penetration is controlled by the relative solvent composition in the polymer solutions used during solvent casting. In one example, the tissue interface layer is comprised of multiple sequentially cast sublayers of chitosan, wherein neighboring layer overlap to about ⅓ of their width. FIG. 28 shows one example of an inter-melding phase (30) between neighbouring layers of chitosan.

In some examples, the tissue interface layer and/or the one or more dosage sublayers each have a thickness in the range of about 50 μm to about 100 μm, and in some examples, each have a thickness in the range of about 60 μm to about 80 μm. In some examples, the tissue interface layer and/or the one or more dosage sublayers each have a thickness of about 60 μm to about 70 μm. In some examples, the spacing sublayers have a thickness in the range of about 0.5 μm to about 311 m, and in some examples, the spacing sublayers have a thickness in the range of about 0.7 μm to about 2.8 μm. In some examples, the backing layer has a thickness of about 5 μm to about 100 μm, and in some examples, a thickness of about 10 μm to about 50 μm.

To facilitate adhesion to the treatment site, one or more of the layers/sublayers may be perforated/fenestrated/porous such that new/healing tissue may grow into or completely through the perforations/fenestrations/pores to thereby anchor the device in place. An adhesive (e.g. tissue glue) or suturing may be used to initially secure the device in place or may be the primary means of securement. In some examples, the backing/support layer may be formed of a polymer matrix robust enough to allow suturing. In some examples, the device may include mounting portions throughout (or “islands”) and/or an outer rim formed of robust materials/polymers configured to provide anchor points for suturing or gluing to the patient. In respect of the post tonsillectomy patient, these anchor points may, for example, be glued or sutured to the tonsillar pillars and/or tonsillar fossa bed, and typically allow for fixation strong enough to resist the forces of swallowing etc. In one example, the mounting portions are formed of any one of a combination polymers selected from the group of: PLLA, PLGA, a copolymer of PLLA and PLGA, and PCL.

Typically, the backing or a sublayer thereof may not be porous, or may have minimum porosity, so as to substantially prohibit/limit diffusion/escape of therapeutic agent from the dosage layers through/across the backing layer, to areas other than the targeted treatment site.

In some examples, one or more of the layers/sublayers, or the device as a whole, has a porosity or matrix void composition in the range of about 60% to about 90%. In some examples, the pore size is selected to facilitate lymphocyte infiltration, fibroblast proliferation, and/or invasion of new vasculature (without which wound healing would be suboptimal). In some examples, the minimum pore size is in the range of about 3 μm to about 7 μm. In some examples, the maximum pore size is about 500 μm. In some examples, the pore size is in the range of about 90 μm to about 130 μm. In one example, the spacing sublayers have a pore size in the range of about 250 μm to about 500 μm, and in some examples, about 274 μm to about 450 μm. In some examples, therapeutic agent is located within pore/matrix voids of the dosage sublayer(s) and tissue interface layer.

It will be appreciated that, in some examples, pore sizes are configured dependent on respective layer thickness (i.e. small enough so as not to fully penetrate the respective layer in which they are present). It will also be appreciated that different layers may have different pore sizes. In some examples, pore sizes for the device are in the range of 200 nm to 600 nm. In some examples, pore sizes for the device are in the range of 6 μm to 60 μm. In some examples pore sizes for the device are in the range of 60 μm to 120 μm.

The layered drug delivery device may take a variety of forms and may be, for example, a patch, insert, implant, or mesh etc. It will be appreciated that the device may take a generally planar form or a non-planar form. The device is typically biocompatible and biodegradable such that over time, it may degrade/dissolve and be absorbed by the body without any ill effect thereon. Degradation may be facilitated by naturally occurring enzymes, such as, for example, those found saliva. It will be appreciated that in some examples, the device may not be fully biodegradable, and may be removed after a certain period once the therapeutic agent has been administered. In such cases, for example, it may be the backing layer that does not biodegrade, and the backing layer in such examples may therefore be comprised of any suitable material (including non-polymeric materials) provided it is non-toxic. It will be appreciated that the backing layer degradability profile can be altered to suit the intended clinical application. For example, degradation of the outer/backing layer of the device may be desired after the entirety of the loaded drug content has been delivered/released such that intraoral device removal is not required (the device degrades and incorporated into the underlying tissue).

Generally, the device is to be located at or against a treatment site, and, in some examples, the device may be shaped/configured to fit particular treatment sites, cavities, fossae or the like. In some embodiments, the device is convex at the tissue interface layer side and concave at the backing layer side. In some embodiments, the device is shaped to be located against a wall of the tonsillar fossa.

It will be appreciated that the device is typically malleable to allow for conformity to a treatment site. In some examples, to promote curvature and/or conformity to a treatment site (e.g. the tonsillar fossa) the layers of the device may be configured to have different levels of hydrophobicity/hydrophilicity. For example, the tissue interface layer may be configured to be substantially hydrophilic, whilst the backing laying configured to be substantially hydrophobic, such that, on placement at a treatment site like the tonsillar fossa, the tissue interface layer absorbs water and expands, providing a curvature that better conforms to the fossa.

For example, cast polymer layers may have differing respective moisture contents and differing abilities to uptake moisture. This may be utilised in high moisture areas to improve mucoadhesion but also to allow the device to self mould to the wound or tissue surface/shape. This improves the devices handling experience for the surgeon and ultimately improves performance by assisting in providing the best possible coverage/contact of/at the wound site.

For example, chitosan only layers typically have high swellability, blended layers formed of combinations of chitosan and PLLA/PLGA/PCL typically have mild swellability, whereas PLGA/PLLA/PCL layers typically have limited swellability.

It will also be appreciated that the nature of the device provides that it may be trimmed/cut to the required size prior to and/or during insertion/surgery so as to more appropriately fit the anatomy of the patient (e.g. pediatric patient vs adult patient).

The drug delivery device may include a range of different types of therapeutic agents including but not limited to drugs, biomolecules, pharmaceutical compositions, and, more particularly, anesthetic agents, antimicrobial agents, antineoplastic agents, antifungal agents, anti-viral agents, chemotherapeutic agents, immune modulators, surfactants, silver and gold particles, steroidal and non-steroidal anti-inflammatories, growth factors, stem cells, cell growth or differentiation promoting agents, nucleic acids (e.g. DNA/RNA), peptides, proteins or antigens for allergy desensitization therapy etc. Generally, in the treatment of post-operative pain, the therapeutic agent is an anesthetic agent. Example anesthetic agents include bupivacaine hydrochloride, lignocaine hydrochloride, ropivacaine hydrochloride, prilocaine hydrochloride, tetracaine hydrochloride, benzocaine hydrochloride.

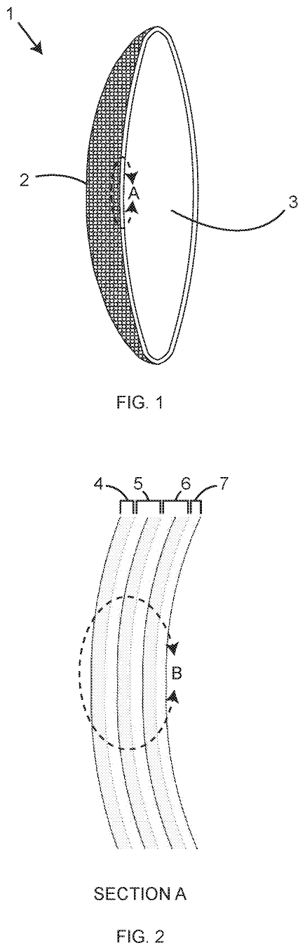

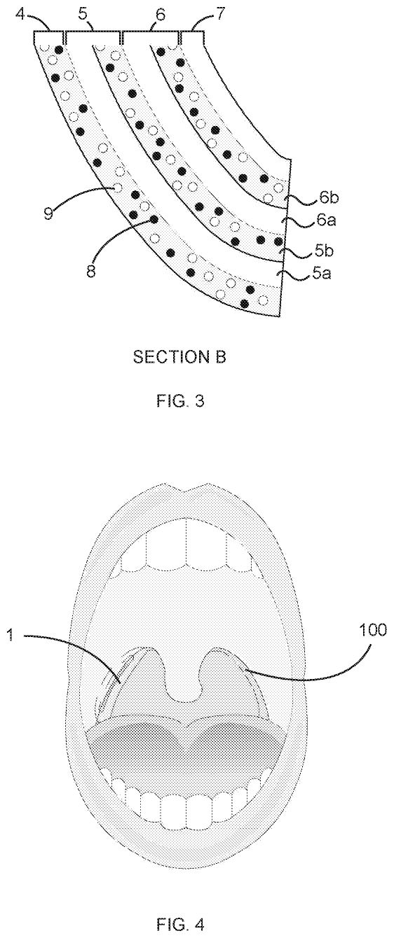

In one particular embodiment, which is illustrated by the schematic diagrams of FIGS. 1 to 4, the invention provides layered drug delivery patch/insert to aid in pain management and wound healing after a tonsillectomy. The patch/insert (1) is shaped to fit the tonsillar fossa (100) and has a generally ovoid shape. The tissue interface side (2) is convex, and the backing side (3) is concave.

The device is multilayered, and includes a tissue interface layer (4), two additional release layers (5, 6), and a backing layer (7). The tissue interface layer (4) is formed of a mixture/blend of chitosan and polycaprolactone (PCL) and has one or more therapeutic agents (8, 9) located/incorporated therein (typically analgesic agents). Each of the additional release layers (5, 6) includes a spacing sublayer (5a, 6a) and a dosage sublayer (5b, 6b). The dosage sublayers are also each loaded with therapeutic agents.

Similar to the tissue interface layer, the dosage sublayers are formed of a combination of chitosan and PCL. The spacing sublayers are formed of a copolymer of poly-l-lactide acid (PLLA) and poly-l-glycolic acid (PLGA).

It will be appreciated that the thickness of the layers/sublayers can vary. In one example of this particular embodiment, the tissue interface layer and dosage sublayers have an average thickness of about 65 μm, the spacing sublayers have an average thickness of about 2.6 μm and the backing layer has a thickness of about 52 μm.

In typical use, post tonsillectomy, the patch/insert (1) is located in the tonsillar fossa over the wound/tissue area to be treated. The mucoadhesive nature of the tissue interface layer (2) and, in particular, the chitosan component thereof, assists with adhesion of the device to the fossa (100) wall. The greater flexibility and swellability of chitosan containing layer at the tissue interface encourages natural adherence and expansion to fit the particular surgical site.

Typically, the device (1) is sutured in place. Alternatively or additionally, in some instances, a glue/adhesive may be used for adhesion to the treatment site. Perforations/fenestrations (2a) in the tissue interface layer (2) also encourage growth of new tissue into the device, to further contribute to secure location in the fossa (100).

In typical use, after the surgeon performs a tonsillectomy, the patch/insert/device (1) is prepared for placement in the tonsillar fossa. The patch/insert device may be provided in multiple sizes to accommodate variation in tonsillar fossa dimensions between patients (e.g. paediatric versus adult patients).

If necessary, the device can be trimmed to fit the tonsillar fossa. The device may, for example, be marked with surgical markers to assist the surgeon in determining the precise size of device necessary, or alternatively, a fitting guide made from inert trans-parent plastic can be used to mark the exact dimensions of the tonsillar fossa. The device is then cut to the appropriate dimensions accordingly.

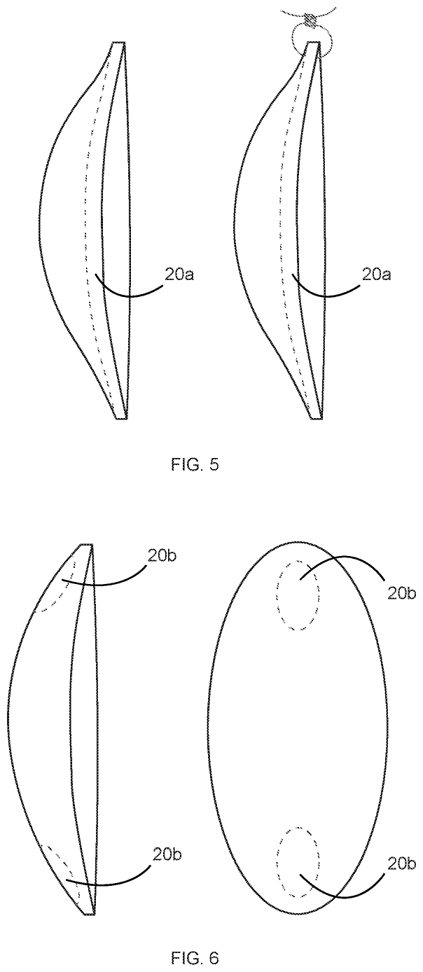

FIGS. 5 and 6 illustrate possible variations in securement method. FIG. 5 shows an example of suturing around an outer rim (20a) of the device, which may be formed of a robust material/polymer (e.g. PLGA, PLLA, a copolymer of PLGA and PLLA or PCL). In this example, the device is typically sutured to the anterior and posterior tonsillar pillars and/or adjacent mucosa. The surgeon may place as many sutures as is their preference to achieve adequate fixation. Is some forms, surgical glue/adhesive may be alternatively applied to the rim (20a).

In the method of FIG. 6, mounting portions or ‘islands’(20b) are topicalised carefully with an appropriate surgical glue and the device then placed in the tonsillar fossa with constant pressure applied until the glue has set and adhesion is adequate. Another variation may be provided whereby glue is pre-incorporated into the polymer of the mounting portions/‘islands’ during manufacture and may be activated by light energy to achieve adhesion to the underlying tissue. It will also be appreciated that a combination of these securement methods may be utilised. The mounting portions/islands typically formed of a robust material/polymer (e.g. PLGA, PLLA, a copolymer of PLGA and PLLA or PCL).

Once secured, therapeutic agents (8, 9) from the tissue interface layer diffuse to the treatment area and/or are released to the treatment area as the tissue interface layer degrades. The neighboring spacing layer (5a), from the adjacent additional release layer (5), provides a barrier/obstacle that delays or slows progression of therapeutic agent from the next dosage layer (5b) to the treatment area/site. Therapeutic agent from the dosage layer (5a) either has to diffuse across the spacing layer and/or is released once the spacing layer has degraded sufficiently. In this respect it will be appreciated how the additional release layers (e.g. 5, 6) provide delayed pulses of therapeutic agent to the treatment site, providing a sustained controlled release of therapeutic. In this example, there are two additional release layers (5, 6) and thus two sequential pulses of therapeutic agent are provided to the treatment site after the initial burst form the tissue interface layer. It will be appreciated that in other forms, the device may include any number of additional release layers, depending on the dosage/release profile required.

The backing layer (7) is configured such that it prohibits/limits diffusion or permeation of therapeutic agent therethrough to other areas of the oral cavity, away from the treatment area. The backing layer (7) includes a sublayer formed of a copolymer of PLLA and PLGA, and a sublayer of PCL which forms the outermost face of the non-tissue facing side of the device (1).

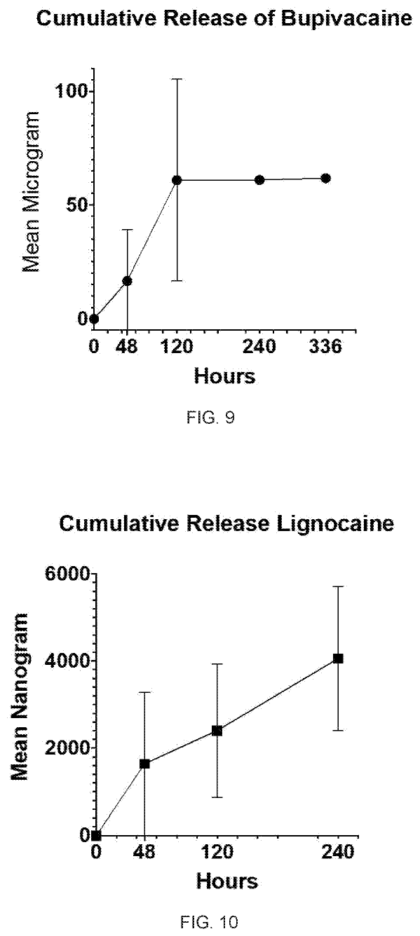

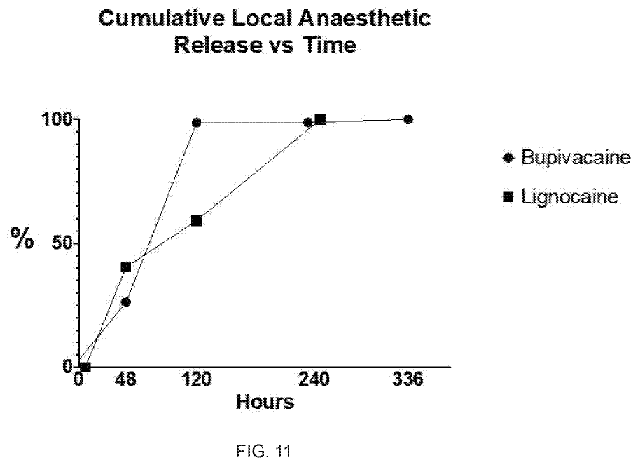

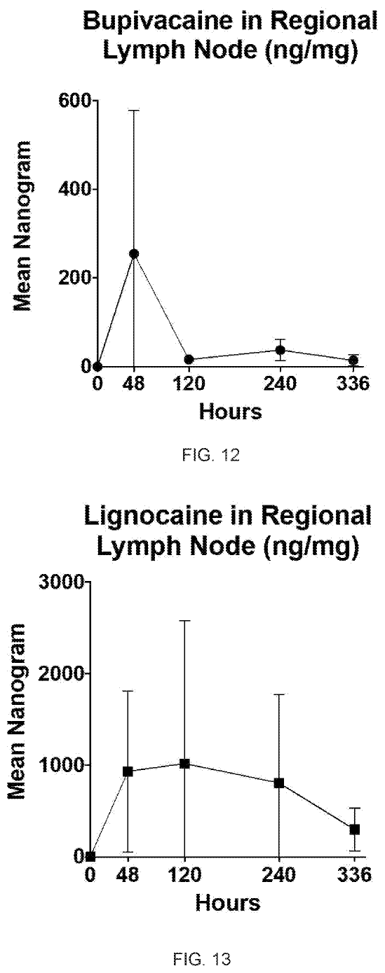

By providing sustained and controlled release of drug/therapeutic, the device allows the patient to avoid any dangerous/toxic spikes in concentration of the administered drug/therapeutic. EXAMPLE 2 and FIGS. 9 and 11 illustrate release profiles achieved with devices in accordance with this particular embodiment, wherein the therapeutic agents is lignocaine and bupivacaine. Corresponding to the release profiles, FIGS. 12 to 15 illustrate levels over time of released therapeutic as detected in the regional lymph nodes and serum.

As a whole, the device (1) is formed of polymer materials that are biodegradable and biocompatible, such that, over time, as it degrades, it is absorbed by the body without ill effect. It will be appreciated that the composition/layers of the device are appropriately configured for the oropharynx so as to be suitably degraded by saliva (e.g. by salivary enzymes) and at the pH of oropharynx (4.0-6.0) or oral cavity (pH 6.0-8.0).

Similarly it will be appreciated that the device (1) has been configured for the oral/pharyngeal environment i.e. to withstand interference from foreign objects (food), the tongue, or throat during swallowing. Chitosan-PCL as well as the copolymers of PLLA an PLGA may be used, like in the above example, due to their more robust strength characteristics so as to prevent device failure during treatment. At the same time chitosan blends may be included, like in the above example, at the tissue interface to maintain levels of moisture interaction, flexibility and the softness required to prevent physical discomfort. It will be appreciated that in other forms, other suitable polymers may be used for tissue interface layers, and additional release layers.

In respect of the backing layer, protection from extreme moisture and an enzyme dense system is required. Here PCL and co-polymers of PLLA and PLGA may be used, like in the above example, for their greater crosslinking and therefore resistance to degradative enzymes and their greater hydrophobic properties to allow the device to perform for longer without failing. It will be appreciated that, in other forms, the backing layer may be formed of other suitable polymers.

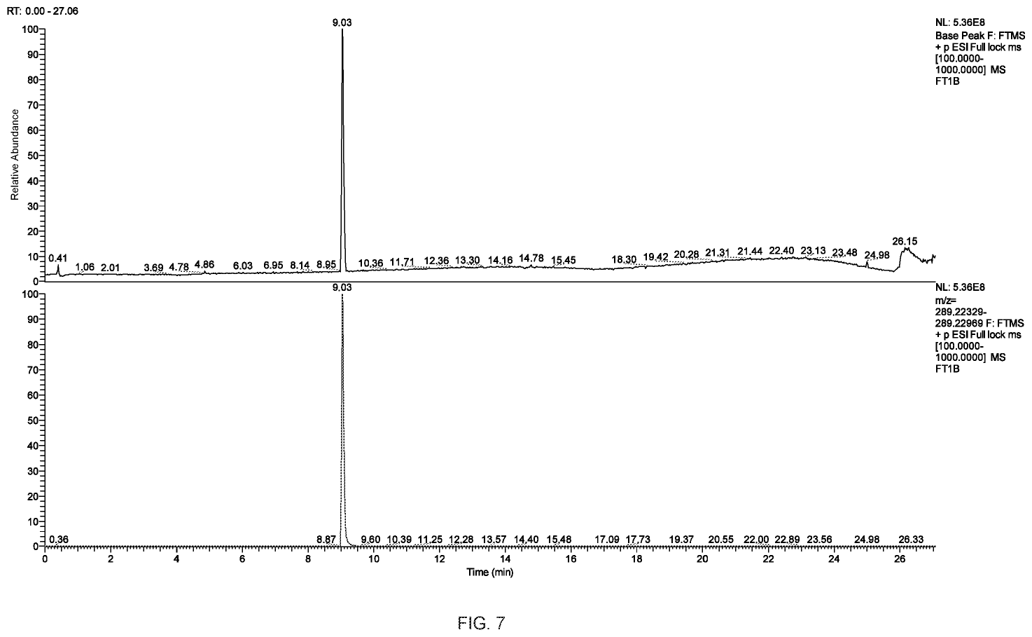

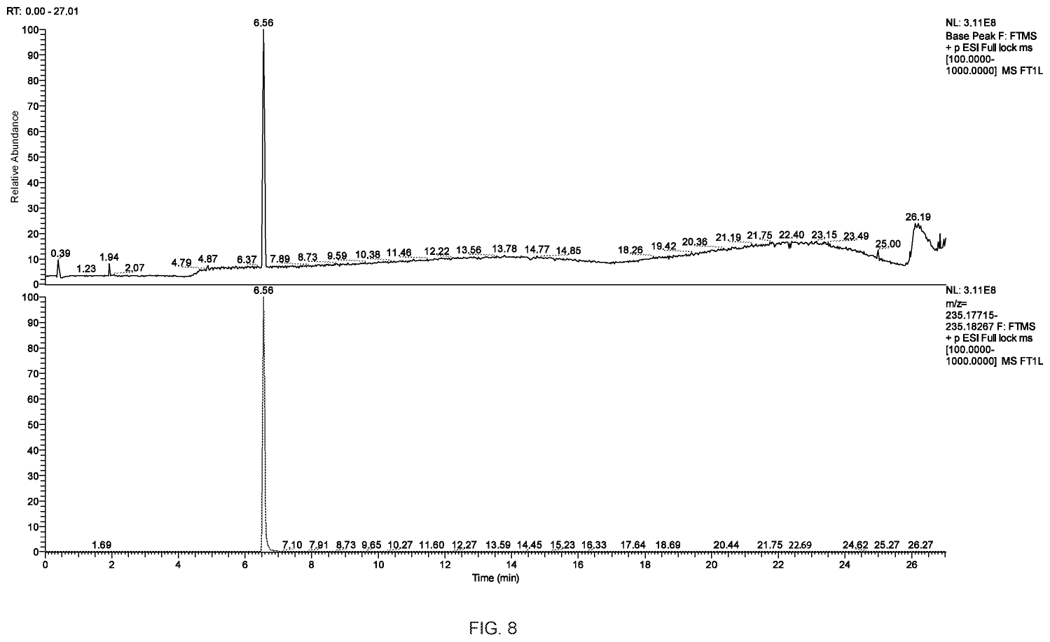

Until release of the active/therapeutic agent (e.g. by degradation of the layers and/or diffusion thereacross) from within the polymer matrix, the drug/therapeutic is preserved and does not degrade from the active form. FIGS. 7 and 8 show examples of chromatograms of released agents (lignocaine and bupivacaine) in one example which indicate that the active form is preserved.

The device (1) and its layers may be produced/fabricated, in one example, in accordance with the methods described in EXAMPLE 1. It will be appreciated that the devices as described herein may be produced using a range of fabrication methods, including injection molding, solvent casting, spray coating, spin coating, electrospraying. In one example one or more of the layers may be injection molded at first instance before subsequent layers are deposited thereon using solvent casting, spray coating or spin coating.

It will be appreciated that for the tonsillectomy patient embodiments of the device may provide a means to:
Deliver local anaesthetic medication to the tonsillar fossa to reduce or eliminate the morbidity of post-tonsillectomy pain;
Augment healing of the tonsillar fossa, expediting remucosalisation and decreasing risk of haemorrhage;
Provide a haemostatic agent in to reduce/limit bleeding;
Provide local anti-microbial effects to prevent infection of the healing wound; and/or
Provide a physical barrier for the healing wound to prevent traumatic removal of eschar.

It will therefore be appreciated that the presently described device may improve patient outcomes post-tonsillectomy by reducing the risk of post-tonsillectomy haemorrhage, optimizing wound healing and decreasing post-operative pain. The sustained analgesic effect reduces patient aversion to eating and drinking post tonsillectomy, in turn reducing the risk of dehydration, weight loss and malnutrition. This leads to reduced clinical dependence on opioids for adequate pain relief and the associated risks of sedation, respiratory depression and death.

It will be appreciated that whilst the above-described particular example relates to a device that is suited for placement in the tonsillar fossa subsequent to a tonsillectomy, the devices as described herein may be shaped/configured for other applications. For example, the devices may be shaped for placement in other areas of the oral cavity, aerodigestive tract, or sinonasal tract. It will also be appreciated devices may be used, and the drug delivery profile adjusted, for any number of surgical procedures including, for example, lingual tonsillectomy, minor malignant and benign oral cavity surgeries, pharyngeal, and laryngeal surgery, major head and neck benign and malignant surgery, uvulopalatopharyngoplasty, adenoidectomy, tongue base channeling, mouth and salivary gland procedures, laryngeal surgery, cleft lip and palate surgery, thyroid surgery, skin wounds and/or dental procedures.

One particular further application relates to Oral/Oropharyngeal Cancer/Robotic Surgery. Transoral robotic surgery is used to perform complex minimally invasive surgical procedures with precision and accuracy, for example in ablative cancer surgery of the throat. These procedures leave open oral/pharyngeal wounds to heal by secondary intention which are painful and like tonsillectomy are accompanied by the risks of bleeding, aversion to oral intake, dehydration, poor wound healing and infection. Ablative wounds vary in size and dimensions based on the extent of the oncological resection required.

In such applications, the device is typically multilayered as described for tonsillectomy application but its physical form/shape can be personalised to fit the intended extent of resection. This can be mapped and templated with preoperative imaging and the device solvent casted to the specified dimensions. This iteration does not strictly come in a concave shape to fit the anatomical space of the tonsillar fossa rather is customised to fit the contour of the defect. This iteration is primarily involved in one-way release of local anaesthetic medication, growth factors or steroid medication for the control of pain, to expedite wound healing and remucosalisation and promote oral intake post-surgery. Furthermore antineoplastic agents such as cisplatin or 5-fluoruracil may be delivered postoperatively from the device to treat microscopic disease or radiosensitise the tissue for external beam radiation thereby reducing the required dose of radiotherapy or maximising its effect.

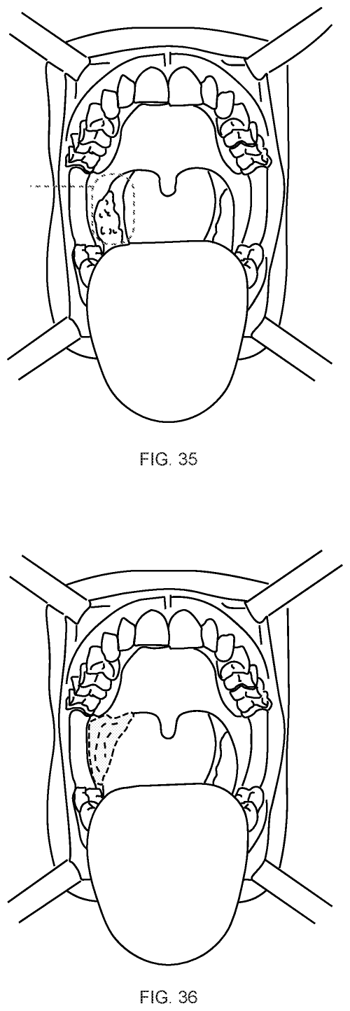

FIGS. 35 and 36 illustrate a schematic of an extended oropharyngeal resection of a tonsillar cancer and oropharyngeal resection defect to which the shape and contour of the device may be customised.

It will also be appreciated that the device is not limited to the application in internal treatment sites (e.g. in the mucosa of body cavities), and may be configured for placement externally, e.g. on the skin to treat external wounds etc. It will also be appreciated that the device may be suitable for the treatment of humans as well as other animals.

Further broad embodiments of the invention relate to a drug delivery device including a polymeric tissue interface layer that includes at least one therapeutic agent, a backing layer, and, optionally, one or more intermediate layers sandwiched there-between. As discussed above, the drug release profile can be modified by appropriately configuring the layer arrangements, number of layers, and layer compositions. The tissue interface layer may, for example, be formed of multiple sequentially cast sublayers that interpenetrate one another. In one example, they interpenetrate by about 25-35% (as a proportion of their width). In one example, the tissue interface layer be formed of multiple sequentially cast layers of chitosan that interpenetrate one another. In one example, the tissue interface layer comprises 4 sequentially cast layers of chitosan that interpenetrate one another (i.e. with 3 intermelded/overlapping phases). In one example, the tissue interface layer for this and other forms may be fabricated in accordance with EXAMPLE 3. One or more of the intermediate layers may be formed of blends of two or more polymers, such as, for example, combinations of any two or more of chitosan, PCL, PLLA, PLGA. Some or more of the intermediate layers may include at least one therapeutic agent. In one example, blended layers for this and other forms may be produced in accordance with EXAMPLE 4. The backing layer may or may not be polymeric, although typically, it is polymeric. In one example, the backing layer may be formed of a combination of any two or more of chitosan, PCL, PLLA, PLGA. In one example, the backing layer for this and other embodiments may be produced in accordance with EXAMPLE 5. It will be appreciated that the therapeutic agent may be incorporated into the tissue interface layer and/or intermediate layers by a range of techniques. As per EXAMPLEs 3 to 5, these may be added to the polymer solutions prior to casting, or alternatively, in accordance with Example 6, included as part of or encapsulated within polymer packets (e.g. for stabilization to preserve the active form).

It is clear that the above-described layered drug delivery devices provide several advantages over prior methods for pain management and wound care. In particular, as the device is adhered to the treatment site, there is no need for repeated oral dosing of medication. Therapeutic agent is rather delivered automatically, in stages or continuously, in accordance with a pre-engineered drug delivery profile. There are therefore no issues with patient compliance. Furthermore, the patch like nature of the device assists with wound healing and the capability to deliver different types of therapeutics allows the delivery of antimicrobial agents (as well as anesthetic agents), so as to reduce the risk of post-operative infection and associated haemorrhage.

It will also be appreciated that according to a further aspect, the present invention provides a unique tissue interface for a drug delivery device. The interface comprising multiple inter-penetrating polymeric layers, typically formed of chitosan.

It will also be appreciated according to a further aspect, the present invention provider unique methods for treating oropharyngeal or tonsillectomy wounds, by utilizing the devices as described herein.

Where ever it is used, the word “comprising” is to be understood in its “open” sense, that is, in the sense of “including”, and thus not limited to its “closed” sense, that is the sense of “consisting only of”. A corresponding meaning is to be attributed to the corresponding words “comprise”, “comprised” and “comprises” where they appear.

While particular embodiments of this invention have been described, it will be evident to those skilled in the art that the present invention may be embodied in other specific forms without departing from the essential characteristics thereof. The present embodiments and examples are therefore to be considered in all respects as illustrative and not restrictive, and all modifications which would be obvious to those skilled in the art are therefore intended to be embraced therein.

### Example 1—Device Fabrication

### Synthesis of Polycaprolactone/Chitosan Drug/Biomolecule Delivery Matrix (for Tissue Interface Layer and Dosage Sublayers)

Polycaprolactione pellets were immersed in 10% v/v acetic acid and 50% w/v citric acid solution at a concentration of 5% w/v and brought to 100-120° C. and mixed for 6 hours until dissolved. The solution was then diluted with deionised water at a concentration of 14-15% v/v and chitosan (medium molecular weight) was then added at a concentration of 1.25% w/v and mixed at 100-120° C. for 2 hours then allowed to cool and mix for a period of 48 hrs. The resultant PCL-Chitosan ratio is 1:2.

### Synthesis of PLLA/PLGA Copolymer Mix (for Backing Layer and Spacing Sublayers)

Poly (L-lactide) pellets were immersed in a 1 dichloromethane: 12 chloroform solution at a concentration of 0.055% w/v and mixed at room temperature for 48 hrs.

### Synthesis of Polycaprolactone Barrier (for Backing Outer Sublayer)

Polycaprolactione pellets were immersed in acetic acid at a concentration of 10% w/v and brought to 100-120° C. and mixed for 6 hours until dissolved.

### Drug Incorporation

Anaesthetic agents such as, but not limited to, bupivacaine hydrochloride, lignocaine hydrochloride, ropivacaine hydrochloride, prilocaine hydrochloride, tetracaine hydrochloride, benzocaine hydrochloride are mixed with the polycaprolactone-chitosan polymer blend at a concentration of 0.005%-0.24% v/v at room temperature and left to mix for 24 hrs.

### Method of Solvent Casting

Starting with the backing layer, PLLA/PLGA copolymer mix was poured into an appropriately shaped glass cast at a volume per surface area ratio of 0.23 ml/cm² at room temperature in an evaporation hood and the solvent allowed to evaporate for 24 hrs.

Drug/biomolecule loaded PCL-Chitosan hydrogel was carefully poured over the PLLA/PLGA backing layer at a volume per surface area ratio of 0.35 ml/cm² at room temperature to cover the underlying backing layer. This hydrogel was then placed at 37° C. in a temperature controlled hood to allow evaporation of solvents for 48 hrs.

This process is repeated two more times with the subsequent PLGA/PLLA sublayers (spacing layers) poured at a volume per surface area ratio of 0.06 ml/cm² and PCL-Chitosan layers at a volume per surface area ratio of 0.35 ml/cm².

To increase rigidity of the backing layer 0.1-0.2 ml 10% v/v polycaprolactone in acetic acid can be placed onto the concave aspect of the device and allowed to dry at room temperature to increased hardness and facilitate suturing (backing outer sublayer).

Fenestrations can be achieved by casting over a preshaped mould whereby the polymers settle around the mould and created a fenestrated device.

The result is a multilayered drug delivery device that includes 3 layers of drug delivery PCL-Chitosan matrix (tissue interface layer+2 dosage sublayers) with PLLA/PLGA copolymer intermediate layers (2 spacing sublayers) to assist in control of one-way drug delivery to the treatment site.

### Example 2—Device Analysis (Tonisillar Application)

Devices produced in accordance with the methods of EXAMPLE 1 were implanted into tonsillectomized pigs.

Samples were taken from the tonsillectomized pigs that were sacrificed at 48, 120, 240 and 336 hours. At necropsy the tonsillar tissue underlying the device was carefully excised, snap frozen and stored at −80 degrees Celsius. Tissue was then freeze ground and small amounts (20-100 mg) stored in individual Eppendorf tubes. The samples were then immersed in methanol for a period of 24 hrs to allow the drug inside the tissue to extract into the solvent and the samples centrifuged to separate solid and liquid com-ponents. The extraction fluid was then analyzed using the following LCMS method.

### HPLC Analysis

Each sample was analysed using a highly sensitive and highly selective bioassay of bupivacaine and lignocaine by liquid chromatography-ion trap mass spectrometry (LC-MS-MS) to detect concentration of drug from samples. The specific LCMS method used for detection of Bupivacaine and Lidocaine has been validated in work by Hoizey et al (2005) (Hoizey G, et al. Sensitive bioassay of bupivacaine in human plasma by liquid-chromatography-ion trap mass spectrometry. Journal of pharmaceutical and biomedical analysis. 2005; 39:587-92).

### Internal Standard Solutions

The methods outlined by Hoizey et al (2005) were employed in analysis of our samples. Validation was repeated at our institution to calibrate our machinery to this method.

Bupivacaine and lignocaine (internal standard) hydrochlorides were purchased from Sigma Aldrich Inc, (Merck, Darmstadt, Del.). Organic solvents and reagents were all of analytical grade. Acetonitrile, diethyl ether, methanol and formic acid were supplied by Sigma Aldrich Inc. Purified water was prepared on a ‘Milli-Q’ water purification system to ensure no signal interference from other ionic compounds or minerals.

Biosamples and Internal Validation

Simulated saliva fluid created from phosphate buffered saline (pH 7.0) with human alpha amylase were used as standard solutions. These standard solutions were evaporated to dryness under a nitrogen stream at 40° C. and dissolved in 200 L of 0.1% formic acid: acetonitrile (50:50 v/v), and 10 L were injected into the LC column.

### Calibration Curve Methods

Stock standard solutions of bupivacaine, lignocaine and respective internal standards (IS) were prepared in methanol at a concentration of 1 mg/mL, and stored at +4° C. These were further diluted in methanol to give appropriate working solutions used to prepare the calibration solutions. Standard curves were prepared in the blank simulated saliva fluid (100 μL) to yield final concentrations of 3.90, 7.81, 15.63, 31.25, 62.5, 125, 250 and 500 μg/L. Once this method was able to be reliably repeated testing pro-gressed to experimental samples.

### Qualitative Sample Analysis (FIGS. 7 and 8)

Qualitative sample analysis (or Q1 test) was performed on select samples to assure single spikes were detected at frequencies consistent with calibration curves and that no secondary spikes were detected (indicating LCMS detection of single molecules without breakdown products).

### Quantitative Sample Analysis (FIGS. 9 to 15)

Each sample was analysed using a highly sensitive and highly selective bioassay of bupivacaine by liquid chromatography-ion trap mass spectrometry (LC-MS-MS) to detect concentration of drug from samples. The specific LCMS method used for detection of Bupivacaine and Lidocaine has been validated in work by Hoizey et al (2005).

FIGS. 9 and 10 represent bupivacaine and lignocaine levels detected by the described LCMS method in porcine tonsillar tissue taken at necropsy at 0, 48, 120, 240 and 336 hrs. Bupivacaine levels are expressed in micrograms and lignocaine in nanograms. These release kinetic curves demonstrate controlled sustained release from the device described in example 1 to the tonsillar tissue interface.

FIG. 11 represents cumulative percentage release of bupivacaine and lignocaine detected by the described LCMS method in porcine tonsillar tissue taken at necropsy at 0, 48, 120, 240 and 336 hrs. These release kinetic curves demonstrate controlled sustained release from the device described in example 1 to the tonsillar tissue interface.

FIGS. 12 and 13 represent bupivacaine and lignocaine levels detected by the described LCMS method in porcine lymph tissue taken from the anterior jugular chain at necropsy at 0, 48, 120, 240 and 336 hrs. Bupivacaine and lignocaine levels are expressed in nanograms per milligram of lymph tissue. These release kinetic curves demonstrate safe levels of the drug detected in locoregional tissue well below the toxic levels of 4 microgram per ml (bupivacaine) and 5.6 microgram per ml (lignocaine).

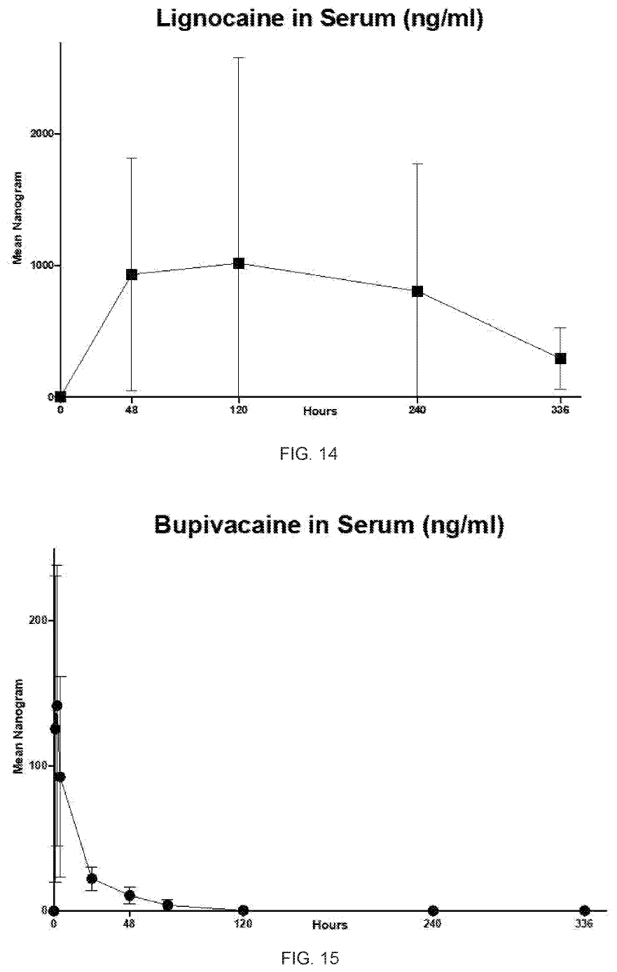

FIGS. 14 and 15 represent bupivacaine and lignocaine levels detected by the described LCMS method in porcine serum taken from the internal jugular vein at 0, 1, 2, 4, 24, 48, 72, 120, 240, 336 hrs. Bupivacaine and lignocaine levels are expressed in nanograms per millilitre of serum. These release kinetic curves demonstrate safe systemic uptake of the drug detected well below toxic levels of 4 microgram per ml (bupivacaine) and 5.6 microgram per ml (lignocaine).

### Scanning Electron Microscopy

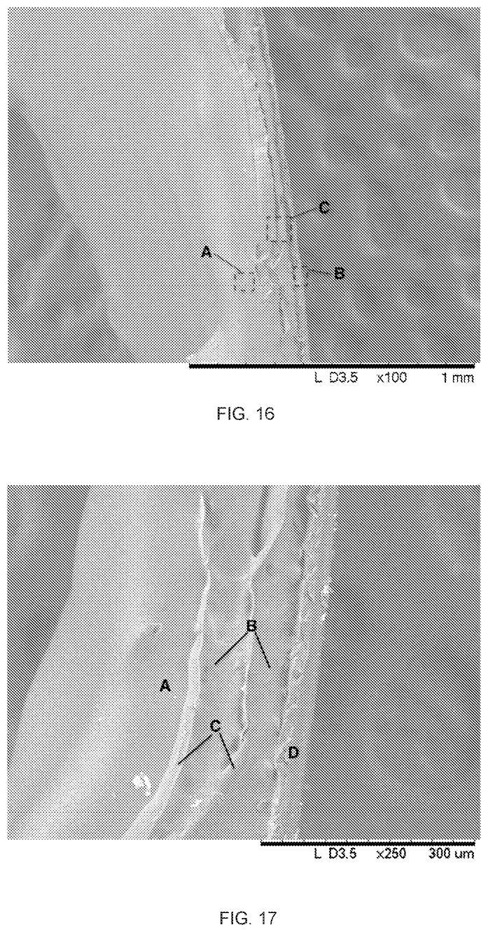

FIGS. 16 and 17 demonstrate scanning electron microscopy (SEM) images of the device in example 1 at ×100 and ×250 magnification respectively. The images illustrate the profile of the device with A representing the interface layer of drug loaded poly-caprolactone-chitosan.

In FIG. 16, B represents the backing or ‘luminal’ aspect of the device made of PLLA:PLGA with a polycaprolactone outer layer to inhibit drug release into the oral cavity/oropharynx. In FIG. 16, C represents the intermediate layers made of PLLA:PLGA designed to slow drug release from the backing layer to the interface layer.

In FIG. 17, B represents intermediate drug delivery layers of PCL/chitosan designed to delivery secondary and tertiary pulses of drug delivery in a unidirectional fashion towards the interface layer. In FIG. 17, C represents the intermediate layers made of PLLA:PLGA designed to slow down drug release pulses from the intermediate layers to the interface layer thereby achieving controlled sustained release of the therapeutic agent.

### Surgical Analysis

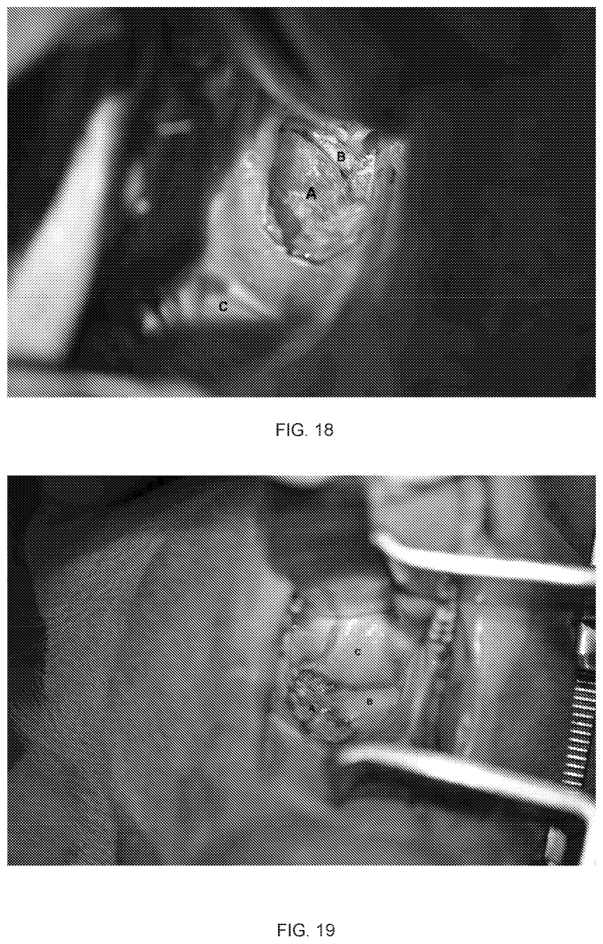

FIG. 18 demonstrates an intraoral view of the device described in example 1 placed in a porcine tonsillectomy wound at time of implantation. The animal is supine with a tonsillectomy gag placed for access to the oropharynx. A is the device with the backing layer visible on the intraluminal aspect. B is a component of the wound created by tonsillectomy. C is the hard palate.

FIG. 19 demonstrates an intraoral view of the device described in example 1 sutured to the porcine tonsillectomy wound 5 days from implantation. The animal is supine with a tonsillectomy gag placed for access to the oropharynx. The device remains adherent to the wound at day 5. A is the device with the backing layer visible on the intraluminal aspect. B is adjacent tonsillar tissue. C is the tongue being retracted by a tongue depressor.

### Histology

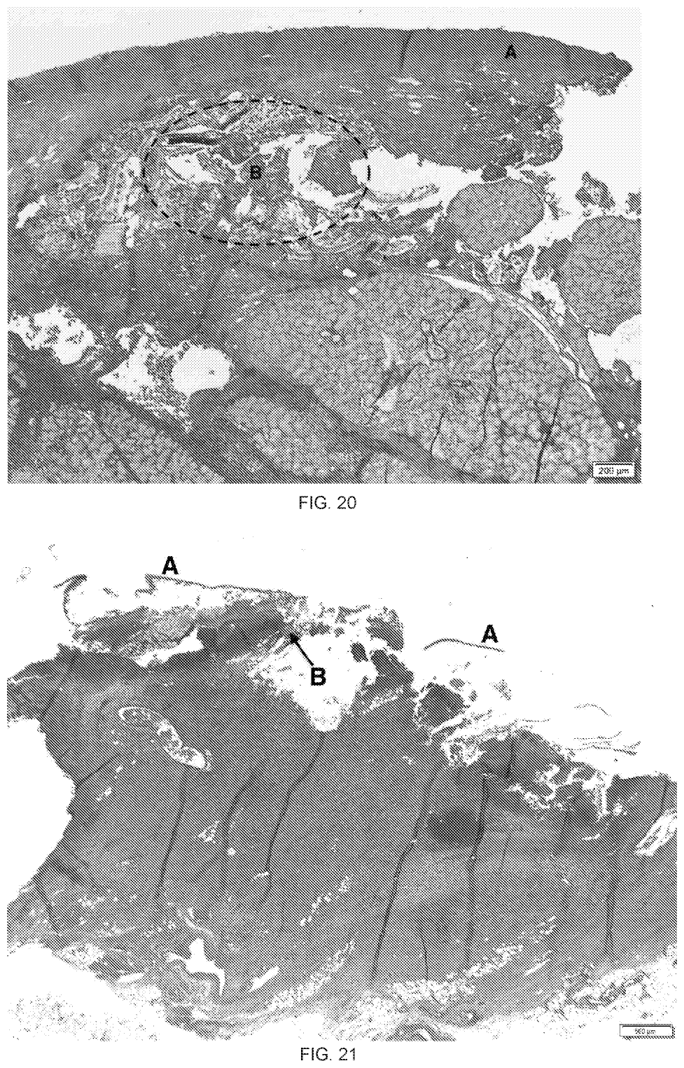

FIGS. 20 to 27 demonstrate tissue responses at the device-tissue interface. These slides were prepared from tissue harvested from porcine tonsillectomy samples at necropsy at time 48, 120, 240 and 336 hours. The entire tonsillectomy wound including underlying muscle was excised with the implant and fixed with formalin 10%. Samples were sliced perpendicular to the plane of device placement to achieve cross sectional images of the device with underlying tissue. Each slide was prepared using H+E staining techniques.

FIG. 20 is a histology slide image of tissue-device interface at 48 hrs. Early granulation tissue at interface (B). Mucosa (A) adjacent to the tissue/polymer interface.

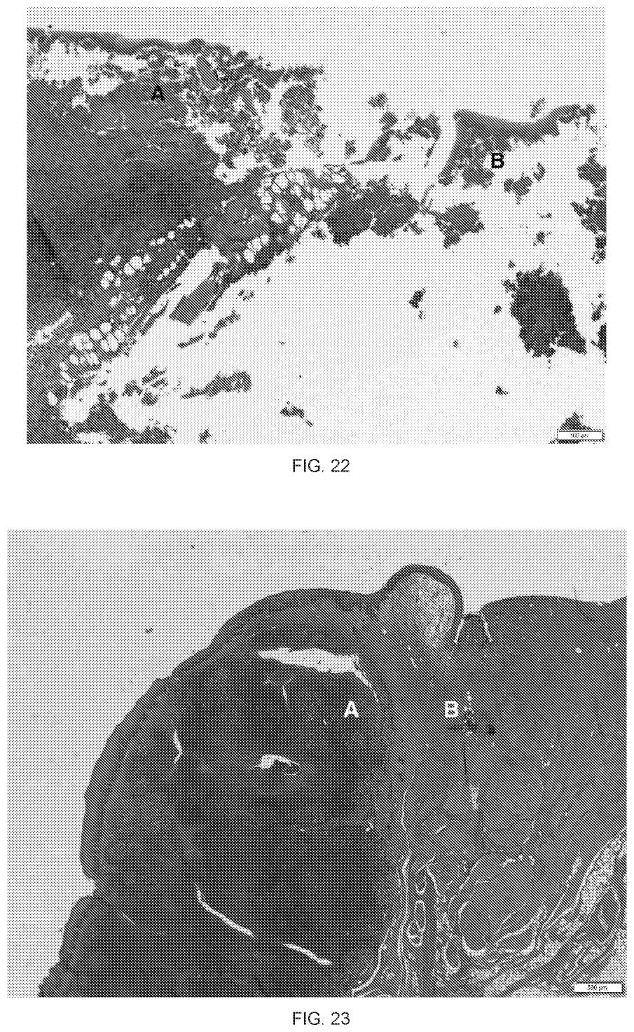

FIGS. 21 and 22 are histology slide images of interface at 5 days (all H+E stains). In FIG. 21, polymer (A) seen with normal granulation tissue (B) (lymphocytes and fibroblasts with early contraction of the wound). FIG. 22 shows a high-power view of granulation tissue (A) at polymer/wound interface (B) with ingrowth of granulation tissue into polymer substance.

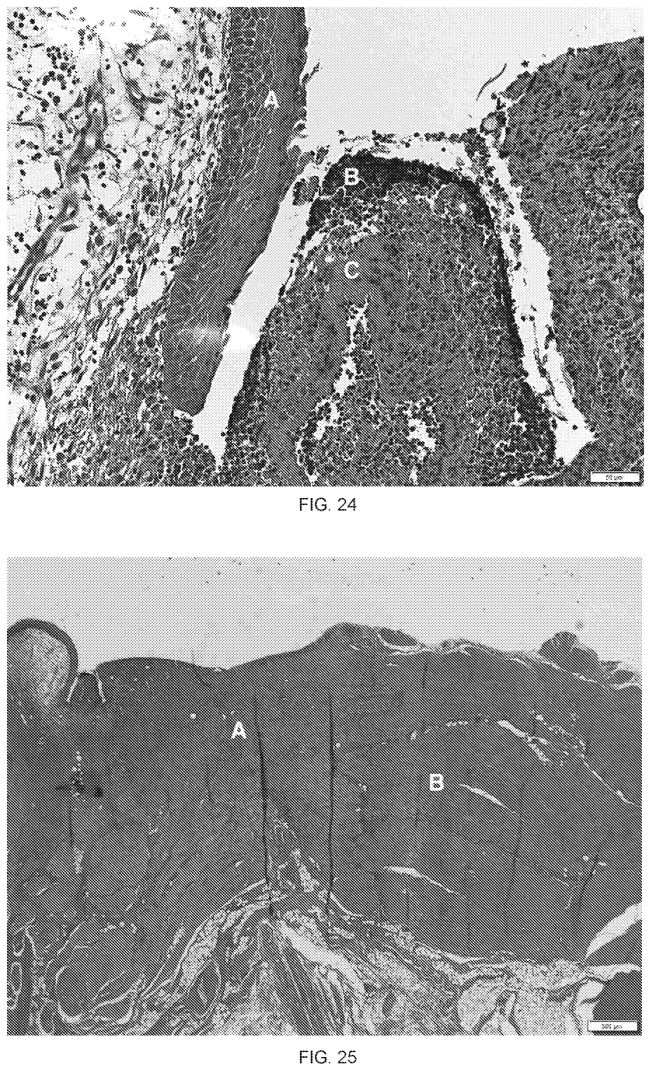

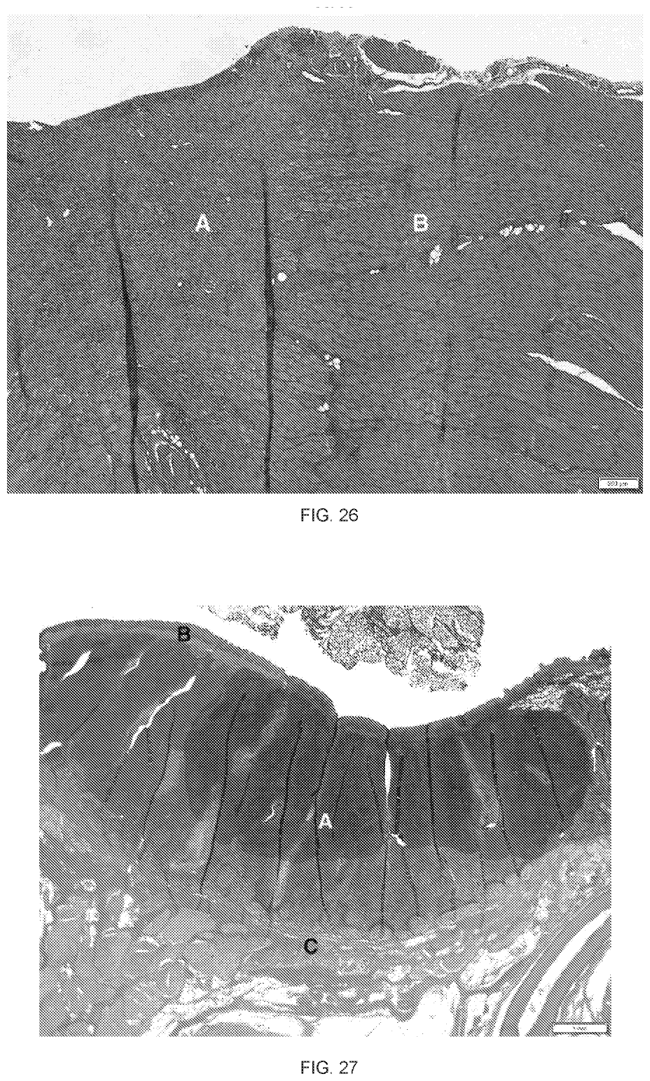

FIGS. 23 to 26 are histology slide images of interface at 10 days. In FIG. 23, a junction between adjacent lymphoid tissue (A) and contracting wound (B) is shown. FIG. 24 shows a high-power field demonstrating adjacent lymphoid tissue and contracting wound with=squamous epithelium (A), lymphocytic infiltrate (B) and newly formed fibrous tissue (C). In FIG. 25, a junction of granulation tissue (A) and contracting fibrous tissue with muscle (B) is shown. FIG. 26 shows a high-powered field of the junction of granulation tissue (A) and contracting fibrous tissue with muscle (B).

FIG. 27 is a histology of interface at 14 days, showing complete healing of tonsillar fossa with new lymphoid tissue (A), squamous epithelium (B) and newly formed fibrous capsule (C).

### Example 3—Fabrication of Tissue Interface Comprising Inter-Melded Chitosan Sublayers

Examples of the device include a tissue-polymer interface or tissue interface layer. This interface may be in the form of one or more chitosan layer(s) with physical properties optimised for tissue interaction. Layer properties, for example, may include one or more of the following:

1. Thickness—(typically 20 μm, or with the range of 10-30 μm)

2. moisture content—(typically 9.5%, or with the range of 5-15%)

3. moisture uptake—(typically 88%, or with the range of 70-95%)

4. porosity—(typically 12%, or with the range of 5-25%)

5. flatness—(typically 100%, or with the range of 90-100%)

6. elasticity—(typically 12%, or with the range of 5-35%)

7. crystallinity—(typically 8.5%, or with the range of 5-20%)

8. tensile strength—(typically 50 MPa, or with the range of 35-75 MPa)

9. surface pH—(typically 7.2, or with the range of 6.8-7.8)

10. water contact angle—(typically 102°, or with the range of 85-110°)

11. surface roughness—(typically 0.07 μm, or with the range of 0.05-0.20 μm)

12. electrical conductivity—(typically Nil, or with the range of Nil)

Further surface modifications may also be included, such as, surface pH modification, chemical surface ionisation, chemical or plasma resurfacing.

An example method of fabrication of a tissue interface comprising inter-melding or interpenetrating chitosan layers is as follows:

Chitosan-tissue interface layers were fabricated following a modified solvent-casting method and were refined by including sintered glass filtration and the solutions pH corrected to approximately 5.0 prior to casting.

Under clean conditions, medium molecular weight Chitosan (190-300 KDa and >85% DDA) was dissolved at 2% (w/v) in a stock aqueous solvent solution. The stock solvent solution contained 97.75% MilliQ water, 2% (v/v) glacial acetic acid, 0.25% (v/v) citric acid. However, the solution may contain an additional 0.05% (v/v) lactic acid.

The gelatinous solution was sealed from the atmosphere and constantly stirred for 48 h at room temperature (25° C.) and then refrigerated at 4° C. for 24 h. The chitosan solution was centrifuged (15 min. 15,000 g) to separate undissolved particulates. Vacuum filtration through a sintered funnel removed smaller undissolved particulates using a glass medium with pore size 35 μm. Under constant stirring, the solution was adjusted to pH 5.0 using a pH probe and drop wise addition of 2 M NaOH. At this stage therapeutics are added such as lignocaine hydrocholride (1% or 2% solutions) and bupivacaine (0.25% or 0.5% solutions).

The polymer solution(s) were then cast onto a sterile-plastic medium at a density of 0.095-0.110 ml/cm2. Polymer layers were formed via solvent evaporation in a sterile laminar flow at room temperature (25° C.) for approximately 14 days.

The solvent casting process was repeated as necessary to gain the desired number of melded phases between polymer additions. The degree to which polymer layers produced transitions or melded phases was controlled via surface ionisation. Each sample washed twice with 0.01 M NaCl and dried before each polymer addition. This gave a suitable transitional phase depth of approximately 25-35% of the previous polymer addition.

In this way the first addition was about ⅔ body, ⅓ upper transition. While the second addition was ⅓ lower transition, ⅓ body, and ⅓ upper transition. This repeats with all additions possessing three phases until the final addition which is the reverse of the bottom addition. I.e. ⅓ lower transition, and ⅔ body.

### Example 4—Fabrication of Blended Layers/Intermediate Layers

Examples of the devices as described herein may include one or more layers containing two or more blended polymers. These may inlcude for example combinations of chitosan, PCL, PLLA, or PLGA. These blended layers may also carry a loading of one or more therapeutic agent, either together or interchangeably. These layer may be imple-mented, in one example, in combination with the those produced in EXAMPLE 3 and 5.

An example method for forming blended layers of Chitosan with PCL, PLLA or PLGA is as follows:

Under clean conditions, medium molecular weight Chitosan (190-300 KDa and >85% DDA) was dissolved at 2% (w/v) in a stock aqueous solvent solution. The stock solvent solution contained 97.75% MilliQ water, 2% (v/v) glacial acetic acid, 0.25% (v/v) citric acid. However, the solution may contain an additional 0.05% (v/v) lactic acid. To a separate solution poly caprolactone, polylactic acid, or polylactic-co-glycolic acid was also added to 10% (w/v) glacial acetic acid and 50% (w/v) citric acid. The solution may contain an additional 0.05% (v/v) lactic acid. These solutions were then mixed.

The gelatinous solution was sealed from the atmosphere and constantly stirred for 48 h at room temperature 100-120° C. and then refrigerated at 4° C. for 24 h. The chitosan polymer blend solution was centrifuged (15 min. 15,000 g) to separate undissolved particulates. Vacuum filtration through a sintered funnel removed smaller undissolved particulates using a glass medium with pore size 35 μm. Under constant stirring, the solution was adjusted to pH 5.0 using a pH probe and drop wise addition of 2 M NaOH. At this stage therapeutics are added such as lignocaine hydrocholride (1% or 2%) or bupivacaine hydrochloride (0.25% or 0.5%) as aqueous solutions.

The polymer solution(s) were then cast onto existing samples at a density of 0.095-0.110 ml/cm2. Polymer layers were formed via solvent evaporation in a sterile chemical fume food at room temperature (25° C.) for approximately 14 days.

The solvent casting process was repeated as necessary to gain the desired number of polymer layers. Each sample washed twice with 0.01 M NaCl in 70% ethanol and dried before each solvent casting.

### Example 5—Backing Layer/Oral Cavity Interface

Example of the device may include includes an oral cavity-polymer interfacing layer or backing layer. This interface may be in the form of one or more chitosan layer(s) with physical properties optimised for interacting with and withstanding complications re-lated to the oral cavity environment.

Such challenges may include but not be limited to:

High sheer stresses, friction, torque and elasticity

Damage due to foreign bodies

High bio load, abundance of degradative enzymes

Extremely high moisture content

In respected to the challenges of the devices desired environment/location the interfacing layer may include but not be limited to, one or more of the following unique properties.

Examples may include one or more layers of a polymer layer, or blended polymer layer, containing one, two or more polymers. Suitable polymers may include, for example, chitosan, PCL, PLLA, or PLGA. These layers may be implented, in one Example in combination with those as provided in EXAMPLES 3 and 4.

The properties may include one or more of the following: 1. Thickness—(typically 15 μm, or with the range of 10-30 μm) 2. moisture content—(typically 10%, or with the range of 5-15%) 3. moisture uptake—(typically 15%, or with the range of 10-25%) 4. flatness—(typically 100%, or with the range of 90-100%) 5. elasticity—(typically 12%, or with the range of 5-35%) 6. tensile strength—(typically 80 MPa, or with the range of 55-95 MPa) 7. surface pH—(typically 7.2, or with the range of 6.8-7.8) 8. water contact angle—(typically 90°, or with the range of 65-95°)

Then addition of poly(dimethylsiloxane-co-alkylmethylsiloxane) may also be included to reduced surface roughness and greatly reduce both friction and hydrophilicity.

An example of a fabrication method is as follows:

The method follows that of EXAMPLE 4. Lactic acid is included is added to the solvent mixture at up to 0.2% v/v.

In iterations incorporating poly(dimethylsiloxane-co-alkylmethylsiloxane) both dichloromethane and poly(dimethylsiloxane-co-alkylmethylsiloxane) 0.5% w/v were added to the polymer solution (PCL, PLLA or PLGA outlined in EXAMPLE 4 prior to initial mixing) or painted to the back of the casted polymer.

### Example 6—Polymer ‘Packets’

Examples of the device may not have therapeutic agent additions directly into the polymer solutions prior to solvent casting i.e. as outlined in EXAMPLES 3-5,

For example ‘packets’ of stabilised therapeutic agents may be manufactured and added to any of the polymer solutions, such as, for example, as outlined in EXAMPLE 3-5, or, for example, added to the surface modification steps outlined in EXAMPLES 3-5.

This permits therapeutics regardless of their natural stability to be included in the device as described herein. The position of therapeutic package inclusion can be either within polymer layers, within inter polymer phases, or between polymer layers them-selves.

An example method of incorporation of therapeutics stabilised in polymer packets is as follows:

Polymer solutions prepared for EXAMPLE 5, to the exclusion of chitosan, were spray dried to create polymer particulates.

Spray drier method—Polymer solutions of either PCL, PLLA, PLGA supplemented with Span 40 and DMSO to reduce viscosity and surface tension. Solutions were fed at an inlet temperature of 50° C. into a Buchi Mini Spray Dryer Model B-290 (Buchi Labora-toriums) using pump setting 25, aspirator setting 80, and a spray flow of 350 L/h and a pressure of 30 mm Hg. Particles are collected in the collection chamber with an outlet temperature of 35° C.

Polymer particulates were then mixed with the therapeutic containing chitosan solution as described in Preferred Method 1 until homogenous. Chitosan and therapeutic agent covered polymer particles of either PCL, PLLA or PLGA where then mixed back into a volume of the starting solutions of the respective PCL, PLLA, or PLGA. This solution was then spray dried again at the respective settings outlined above.

These donut like particle containing a stabilised chitosan/therapeutic agent within a protect polymer jacket.

These stabilised particles can then be used to substitute direct additions of ther-apeutic agents, e.g. as described in EXAMPLES 3 to 5 or as an addition to the surface preparation washes outlined in EXAMPLES 3 to 5 which allows deposition of additional therapeutic agents between structural polymer layers.

1. A layered drug delivery patch for securement in the oropharynx, the patch including:
a perforated polymeric tissue interface layer including at least one therapeutic agent;
a polymeric backing layer; and
one or more additional release layers sandwiched between the tissue interface layer and the backing layer, each additional release layer including:
a polymeric spacing sublayer; and
a polymeric dosage sublayer including at least one therapeutic agent,
wherein the sublayers of each additional release layer are ordered such that each spacing sublayer is closer to the tissue interface layer than its respective dosage sublayer.

2. A patch as claimed in claim 2, wherein the device includes at least two additional release layers.

3. A patch as claimed in claim 1, wherein the tissue interface layer is formed of a polymer matrix with therapeutic agent incorporated therein.

4. A patch as claimed claim 1, wherein the dosage sublayer(s) is/are formed of a polymer matrix with therapeutic agent incorporated therein.

5. A patch as claimed in claim 1, wherein the tissue interface layer comprises a polymer matrix formed of a blend of two or more polymers.

6. A patch as claimed in claim 5, wherein the tissue interface layer is formed of a blend of chitosan and PCL.

7. A patch as claimed in claim 1, wherein each dosage sublayer comprises a polymer matrix formed of a blend of two or more polymers.

8. A patch as claimed in claim 7, wherein the dosage sublayer(s) is/are formed of a blend of chitosan and PCL.

9. A patch as claimed in claim 1, wherein the spacing sublayer(s) is/are configured to slow or delay release of therapeutic agent from the dosage sublayers.

10. A patch as claimed in claim 1, wherein the spacing sublayer(s) is/are formed of a copolymer of PLLA and PLGA.

11. A patch as claimed in claim 1, wherein the backing layer is configured to substantially prohibit diffusion or permeation of therapeutic agent therethrough.

12. A patch as claimed in claim 1, wherein the backing layer includes a layer of PCL.

13. A patch as claimed in claim 1, wherein the backing layer includes a sublayer formed of copolymer of PLLA and PLGA, and a sublayer formed of PCL, the PCL sublayer being the outermost layer, furthest from the tissue interface layer.

14. (canceled)

15. A patch as claimed in claim 1, wherein the tissue interface layer comprises two or more sequentially cast polymeric sublayers that interpenetrate one another.

16. A patch as claimed in claim 15, wherein the sequentially cast sublayers of the tissue interface layer comprise chitosan.

17. A patch as claimed in claim 16, wherein the neighboring sublayers interpenetrate one another by about 25-35%, as proportionate to their width.

18. (canceled)

19. A patch as claimed in claim 1, wherein the layers thereof are continuous.

20. A patch as claimed in claim 1, wherein the at least one therapeutic agent includes an anesthetic agent.

21. A method of treating an oropharyngeal wound, the method including the steps of:
securing a patch as claimed in claim 1 against the wound.

22. (canceled)

23. Use of a patch as claimed in claim 1, in the treatment of an oropharyngeal wound.

24. (canceled)
